(19) United States
(12) Patent Application Publication
(10) Pub. No.: US 2024/0249801 A1
(43) Pub. Date: Jul. 25, 2024

(54) CALIBRATING AN ELECTRONIC CHEMICAL SENSOR TO GENERATE AN EMBEDDING IN AN EMBEDDING SPACE

(71) Applicant: Osmo Labs, PBC, Cambridge, MA (US)

(72) Inventor: Alexander Wiltschko, Somerville, MA (US)

(21) Appl. No.: 18/561,610

(22) PCT Filed: May 4, 2022
(86) PCT No.: PCT/US22/27629

§ 371 Date: Nov. 16, 2023

Related U.S. Application Data
(60) Provisional application No. 63/189,501, filed on May 17, 2021.

(51) Int. Cl. G16C 20/70; G01N 33/00; G16C 20/30; G16H 50/50

(52) U.S. Cl. CPC G16C 20/70; G01N 33/0034; G16C 20/30; G16H 50/50

(57) ABSTRACT

Electronic chemical sensors can output raw electrical signal data in response to sensing a chemical compound, but the raw electrical signal data can be difficult to interpret. Processing the electrical signal data with a machine-learned model to generate an embedding output in an embedding space can provide a better understanding of the electrical signal data. Moreover, leveraging preexisting chemical property prediction models to generate other embeddings in the embedding space can allow for more accurate and efficient classification tasks of the electrical signal data.

### RELATED APPLICATIONS

This application claims priority to and the benefit of U.S. Provisional Patent Application No. 63/189,501, filed May 17, 2021. U.S. Provisional Patent Application No. 63/189,501 is hereby incorporated by reference in its entirety.

### FIELD

The present disclosure relates generally to processing sensor data to detect and/or generate representations of chemical molecules. More particularly, the present disclosure relates to generating sensor data, processing the sensor data with a machine-learned model to generate embedding outputs, and using the embedding outputs to perform various tasks.

### BACKGROUND

Computing devices can be used for visual computing or audio processing, but computing devices lack the ability to robustly sense smells. There are chemical sensors available, but they produce raw signals that are challenging to interpret. The chemical sensors cannot convert the raw signals into a human-interpretable label, like ‘orange’, or ‘cinnamon’, across the entire space of possible odors. Some computing devices have been configured to determine a small subset of smells based on individual training, but these computing devices fail to determine non-trained properties.

Moreover, individual training of all possible smells would be time consuming and computationally taxing once finally configured, and even after such training, the combination of known smells would not be able to be determined. Scents would be associated only with inputted data and determining the olfactory properties of new mixtures would not be possible.

### SUMMARY

Aspects and advantages of embodiments of the present disclosure will be set forth in part in the following description, or can be learned from the description, or can be learned through practice of the embodiments.

One example aspect of the present disclosure is directed to a computing system. A computing system can include a sensor configured to generate electrical signals indicative of presence of one or more chemical compounds in an environment and a machine-learned model trained to receive and process the electrical signals to generate an embedding in an embedding space. In some implementations, the machine-learned model may have been trained using a training dataset including a plurality of training examples, each training example including a ground truth property label applied to a set of electrical signals generated by one or more test sensors when exposed to one or more training chemical compounds. Each ground truth property label can be descriptive of a property of the one or more training chemical compounds. The computing system can include one or more processors and one or more non-transitory computer-readable media that collectively store instructions that, when executed by the one or more processors, cause the computing system to perform operations, the operations comprising. The operations can include generating, by the sensor, sensor data indicative of presence of a specific chemical compound in the environment and processing, by the one or more processors, the sensor data with the machine-learned model to generate an embedding output in the embedding space.

In some implementations, the operations can include performing a task based on the embedding output. The task can include providing a sensory property prediction based on the embedding output. In some implementations, the task can include providing an olfactory property prediction based on the embedding output. The task can be identifying a disease state based at least in part on the embedding output. In some implementations, the task can be determining a malodor state based at least in part on the embedding output. The task can be determining if spoilage has occurred based at least in part on the embedding output. The task can include providing a human-inputted label for display, and the human-inputted label can be determined by an association with the embedding output in the embedding space. The human-inputted label can be descriptive of a name of a particular food.

In some implementations, the machine-learned model can be trained jointly with a graph neural network, and training can include jointly training the machine-learned model and the graph neural network to generate a single, combined output within the embedding space. The graph neural network can be trained to receive a graph-based representation of the specific chemical compound as an input and output a respective embedding in the embedding space.

In some implementations, the machine-learned model may have been trained by obtaining a chemical compound training example comprising electrical signal training data and a respective training label. The electrical signal training data and the respective training label can be descriptive of a specific training chemical compound. The machine-learned model may have been trained by processing the electrical signal training data with the machine-learned model to generate a chemical compound embedding output: processing the chemical compound embedding output with a classification model to determine a chemical compound label: evaluating a loss function that evaluates a difference between the chemical compound label and the respective training label: and adjusting one or more parameters of the machine-learned model based at least in part on the loss function.

In some implementations, the machine-learned model can be trained with supervised learning. The sensor data can be descriptive of at least one of voltage or current. The machine-learned model can include a transformer model. In some implementations, the operations can include storing the embedding output. The sensor data can be descriptive of an amplitude of one or both of voltage or current for one or more electrical signals. The processing, by the one or more processors, the sensor data with the machine-learned model to generate the embedding output in the embedding space can include compressing the sensor data to a fixed length vector representation.

Another example aspect of the present disclosure is directed to a computer-implemented method. The method can include obtaining, by a computing system including one or more processors, sensor data with one or more sensors. In some implementations, the sensor data can be descriptive of electrical signals generated due to a presence of one or more chemical compounds in an environment. The method can include processing, by the computing system, the sensor data with a machine-learned model to generate an embedding output in an embedding space. The machine-learned model can be trained to receive and process data descriptive of electrical signals to generate an embedding in the embedding space. The method can include determining, by the computing system, one or more labels associated with the embedding output in the embedding space and providing, by the computing system, the one or more labels for display.

Another example aspect of the present disclosure is directed to one or more non-transitory computer readable media that collectively store instructions that, when executed by one or more processors, cause a computing system to perform operations. The operations can include obtaining sensor data with one or more sensors. In some implementations, the sensor data can be descriptive of electrical signals generated due to a presence of one or more chemical compounds in an environment. The operations can include processing the sensor data with a machine-learned model to generate an embedding output in an embedding space. The machine-learned model can be trained to receive and process data descriptive of electrical signals to generate an embedding in the embedding space. The operations can include obtaining a plurality of stored sensory property data sets, in which the plurality of stored sensory property data sets can include stored embeddings in the embedding space paired with a respective sensory property data set associated with the respective stored embedding. The operations can include determining one or more sensory properties based on the embedding output in the embedding space and the plurality of stored sensory property data sets and providing the one or more sensory properties for display.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system that performs sensor data processing according to example embodiments of the present disclosure.

FIG. 1B depicts a block diagram of an example computing device that performs sensor data processing according to example embodiments of the present disclosure.

FIG. 1C depicts a block diagram of an example computing device that performs sensor processing according to example embodiments of the present disclosure.

FIG. 2 depicts a block diagram of example classification processes according to example embodiments of the present disclosure.

FIG. 3 depicts a block diagram of an example electronic chemical sensor system according to example embodiments of the present disclosure.

FIG. 4 depicts a block diagram of an example training process according to example embodiments of the present disclosure.

FIG. 5 depicts a block diagram of an example sensor data machine-learned model processing according to example embodiments of the present disclosure.

FIG. 6 depicts a flow chart diagram of an example method to perform sensor data processing according to example embodiments of the present disclosure.

FIG. 7 depicts a flow chart diagram of an example method to perform sensor data processing according to example embodiments of the present disclosure.

FIG. 8 depicts a flow chart diagram of an example method to perform machine-learned model training according to example embodiments of the present disclosure.

FIG. 9 depicts a block diagram of an example training process according to example embodiments of the present disclosure.

Reference numerals that are repeated across plural figures are intended to identify the same features in various implementations.

### DETAILED DESCRIPTION

### Overview

Generally, the present disclosure relates to processing sensor data descriptive of the presence of chemical molecules. The systems and methods can be used for electrical signal processing to enable the interpretation of sensor data obtained from an electronic chemical sensor device. The systems and methods disclosed herein can leverage a trained machine-learned model to process sensor data to generate embedding outputs in an embedding space that can then be used to perform a variety of tasks. Training of the machine-learned model can use ground truth data sets and may utilize a database of pre-existing chemical molecule property data.

More specifically, in some implementations, the systems disclosed herein can include a sensor configured to generate electrical signals. The electrical signals can be indicative of the presence of one or more chemical compounds in an environment, and a machine-learned model can be trained to receive and process the electrical signals to generate an embedding in an embedding space. The machine-learned model can be trained using a training dataset including a plurality of training examples. The training examples can include ground truth property labels applied to respective sets of electrical signals generated by the sensor when exposed to one or more training chemical compounds. The ground truth property labels can be descriptive of a property of the one or more training chemical compounds. Moreover, the system can include one or more processors and one or more non-transitory computer-readable media that collectively store instructions that, when executed by the one or more processors, cause the handheld remote control device to perform operations. These components can be included to enable the sensor to generate sensor data based on electrical signals which can then be processed with the machine-learned model to generate an embedding output in the embedding space. More particularly, the systems and methods disclosed herein can be used to generate sensor data descriptive of electrical signals generated when chemical features of a sensor react with a chemical compound in an environment. The sensor data can then be processed by the machine-learned model to generate an embedding output in an embedding space. In some implementations, the embedding space can be populated by embeddings generated based on electrical signals and embeddings generated based on graph-representations of chemical compounds. Moreover, in some implementations, the embedding space can be populated with embedding labels descriptive of chemical mixture names or properties, which may be generated based on human-input or automatic prediction.

In some implementations, the systems and methods can further include performing a task based on the embedding output. The task can include providing a classification output, determining property predictions, providing an alert, and/or storing the embedding output. For example, the embedding output may be processed to determine one or more property predictions, which can then be provided for display to a user. The property predictions can be sensory property predictions such as olfactory property predictions or volatility predictions which can be determined and lead to providing a dangerous chemical alert.

In some implementations, the machine-learned model can be trained by obtaining the plurality of training examples, in which the training examples include electrical signal data sets and respective training labels. The training electrical signal data sets and the respective training labels can be descriptive of specific chemical compounds. The electrical signals can be processed to generate embedding outputs. The embedding outputs can then be processed by a classification model to determine a chemical compound label for each respective electrical signal data set. The resulting labels can be compared to the ground truth labels to determine if adjustments to the parameters of the machine-learned model need to be made. Moreover, in some implementations, the machine-learned model may be trained jointly with a graph neural network (GNN) model in order to generate embeddings using graph representations or electrical signals, which can then be used for classification tasks. In some implementations, the training can involve supervised learning.

The trained machine-learned model can then be used for a variety of tasks including predicting properties of a sample based on electrical signals, determining if crops are diseased, identifying food spoilage, diagnosing disease, determining a malodor exists, etc. The machine-learned model can be housed locally on a computing device as part of an electrical chemical sensor device or can be stored and accessed as part of a larger computing system. The systems and processes can be used for individual use, commercial use, or industrial use with a variety of applications.

An electronic chemical sensor can include one or more sensors and, optionally, one or more processors. The device can use the one or more sensors to obtain sensor data descriptive of an environment. The sensor data may be descriptive of the chemical compounds in the environment. In some implementations, the sensor data can be processed to determine a mixture composition. The sensor data can be processed with a machine-learned model to determine the mixture. Determining the mixture can involve processing the sensor data to generate an embedding which can then be processed by a classification model to determine the mixture composition. In some implementations, the determination process can utilize a labeled embedding space generated using labeled embeddings. The determined mixture can be determined based on a determined one or more mixture labels in a labeled embedding space.

Calibrating the electronic chemical sensor device to determine mixtures or properties can include obtaining a plurality of mixture data sets. The mixture data sets can be descriptive of one or more sensory properties for respective mixtures. One or more mixture labels can be obtained for each mixture of the plurality of mixtures. The plurality of mixture data sets can be processed with a machine-learned model to generate a plurality of mixture embeddings. Each mixture embedding can be associated with a respective mixture data set. The plurality of embeddings can then be paired with respective mixture labels. The labeled embeddings can be used to generate the labeled embedding space.

In some implementations, the mixture labels can be human-inputted labels. In some implementations, the system can collect accurate human labeled sensor data for calibration (e.g., human labeled odor data). The calibrated electronic chemical sensor device can then detect chemical matter, composed of a mixture of molecules, where each molecule may be at a different concentration. In some implementations, the one or more sensors can include an electronic nose sensor that can generate the sensor data. The sensor data may be descriptive of electronic signals. The one or more sensors may include, but are not limited to, carbon nanotubes, DNA-conjugated carbon nanotubes, carbon black polymers, optically-sensitive chemical sensors, sensors constructed by conjugated living sensors with silicon, olfactory sensory neurons cultured from stem cells or harvested from living things, olfactory receptors, and/or metal oxide sensors. The resulting sensor data can be raw data including voltage or current data.

In some implementations, an experiment where both human labels and electronic signals can be collected on an identical sample, or an appreciably similar sample, can be used for calibration. In some implementations, the machine-learned model can be trained using ground truth training data comprising a plurality of sensory data sets and the plurality of mixture labels. The machine-learned model may include one or more transformer models and/or one or more GNN embedding models.

Moreover, calibration of the electronic chemical sensor device can include mapping the human labels onto an embedding space (e.g., an odor embedding space). Mapping can utilize a trained GNN. Use of the device can then involve mapping obtained electrical signals onto the embedding space. The mapped location (i.e., embedding space values) can be used to automatically recognize odors or other sensory properties with human labels such as ‘cinnamon’, ‘cucumber’, ‘apple’ and ‘feces’. Mapping of the electrical signals can be performed using a GNN trained on electronic nose signals, using deep neural networks. In some implementations, the embeddings can be configured similar to RGB numbering. In some implementations, processing the sensor data and the embedding space can include processing the sensor data with the machine-learned model to generate an embedding, mapping the embedding in the embedding space, and determining a matching label based on a location of the embedding related to one or more mixture labels.

The accuracy of predicting human labels can be assessed with electronic sensor signals. A low accuracy on a specific human label such as ‘cinnamon’ can indicate the sensor is not able to accurately detect that odor. A high accuracy on a specific label can indicate the sensor is able to accurately detect that odor.

In some implementations, the electronic chemical sensor can be composed of a number of distinct sensing elements, akin to how a camera is able to sense both red and green colors. Using this system of co-collected human labeled data and electronic signal data, the system can assess whether a new sensing element (suppose a camera were now able to sense blue colors) improves the ability to cover the space of odors recognizable by a human, or whether it improves the ability to recognize a specific odor label.

Instead of recognizing a human-defined odor label, the system may instead define the labels as the presence or absence of humans, animals, or plants in a diseased state, which give off characteristic odors.

In some implementations, the systems and methods disclosed herein can be implemented to identify foods or particular flavors based on sensor data collected. For example, a glass of orange juice may be placed below a sensor to generate sensor data descriptive of the exposure of one or more chemicals. The sensor data can be processed by the machine-learned model to generate an embedding output in an embedding space. The embedding output can then be used to determine a food label and/or a flavor label. For example, the embedding output may be determined to be most similar to an embedding paired with an orange label or orange juice label. In some implementations, the embedding output may be analyzed to determine the sensed chemical is indicative of a citrus flavor. Determination of the food type and flavor may involve a classification model, threshold determination, and/or analyzing a labeled embedding space or map.

Another example use of the systems and methods disclosed herein can include the enablement of a diagnostic sensor for human diagnostics, animal diagnostics, or plant diagnostics. The presence of certain chemicals can be indicative of certain disease states. For example, chemical compounds found in the breath of a human can provide valuable information on the presence and stages of certain illnesses or diseases (e.g., gastroesophageal reflux disease, periodontitis, gum disease, diabetes, and liver or kidney disease). Therefore, in some implementations, sensor data can be descriptive of exposure to chemicals exhaled from a mouth or taken as a sample from the patient. The sensor data can be processed by the machine-learned model to generate an embedding output. The embedding output can be compared to embeddings indicative of sensed disease states or may be processed by a classification head trained for diagnostics to determine if chemicals indicative of a disease state are present. The output of the classification head may include probabilities of each of one or more disease states being present.

Electronic chemical sensor devices can be implemented into cooking appliances such as stoves or exhaust hoods to aid in cooking and provide alerts on the cooking process. In some implementations, electronic chemical sensor devices can be implemented to provide alerts that a chemical indicative of burnt food is present. For example, the embedding output may be input into a classification head, which processes the embedding output to determine a probability of burnt food being present. If the probability is above a threshold probability, an alert may be activated.

Moreover, in some implementations, electronic chemical sensor devices with trained machine-learned models can be implemented into agricultural equipment such as ground vehicles and low flying UAVs to detect the presence of diseased crops or to detect if the plants are ripe for harvest. For example, the embedding output may be input into a classification head, which processes the embedding output to determine a probability of that the plants are ripe for harvest.

In some implementations, the systems and methods disclosed herein may be used to control machinery and/or provide an alert. The systems and methods can be used to control manufacturing machinery to provide a safer work environment or to change the composition of a mixture to provide a desired output. Moreover, in some implementations, real-time sensor data can be generated and processed to generate embedding outputs that can be classified to determine if an alert needs to be provided (e.g., an alert to indicate a dangerous condition, food spoilage, a disease state, a bad odor, etc.). For example, in some implementations, the determined classifications may include the property predictions such as olfactory property predictions for the scent of a vehicle used for transportation services. The classification can then be processed to determine when a new scent product should be placed in the transportation device and/or whether the transportation device should undergo a cleaning routine. The determination that a mal odor is present may then be sent as an alert to a user computing device or may be used to set up an automated purchase. In another example, the transportation device (e.g., an autonomous vehicle) may be automatically recalled to a facility to undergo a cleaning routine. In another example, an alert can be provided if a property prediction generated by the machine learning model indicates an unsafe environment for animals or persons are present within a space. For example, an audio alert can sound in a building if a prediction of a lack of safety is generated based on sensed chemicals in the building. As an example, the embedding output may be input into a classification head, which can process the embedding output to determine a probability that the environment contains an unsafe chemical. If the probability is above a threshold probability, an alert may be issued and/or an alarm may be activated.

In some implementations, the system may intake sensor data to be input into the embedding model and classification model to generate property predictions of the environment. For example, the system may utilize one or more sensors for intaking data associated with the presence and/or concentration of molecules in the environment. The system can process the sensor data to generate input data for the embedding model and the classification model to generate property predictions for the environment, which can include one or more predictions on the smell of the environment or other properties of the environment. If the predictions include a determined unpleasant odor, the system may send an alert to a user computing device to have a cleaning service completed. In some implementations, the system may bypass an alert and send an appointment request to a cleaning service upon determination of the unpleasant odor.

Another example implementation can involve background processing and/or active monitoring for safety precautions. For example, the system can actively generate and process sensor data obtained with sensors in a manufacturing plant to ensure the manufacturer is aware of any dangers. In some implementations, sensor data may be generated at interval times or continually and may be processed by the embedding model and classification model to determine the property predictions. The property predictions can include whether chemicals in the environment are flammable, poisonous, unstable, or dangerous in any way. For example, the property predictions may include a probability score for each of a plurality of environmental hazard states being present. If chemicals sensed in the environment are determined to be dangerous in any way, for example if the probability score for any one or more environmental hazard states exceeds a respective threshold value, an alert may be sent. Alternatively and/or additionally, the system may control one or more machines to stop and/or contain the process to protect from any potential present or future danger.

The systems and methods can be applied to other manufacturing, industrial, or commercial systems to provide automated alerts or automated actions in response to property predictions. These applications can include identifying sensed chemicals, determining properties of the sensed chemical, identifying diseases, identifying food spoilage, or determining issues with crops.

In some implementations, the systems and methods disclosed herein can leverage a chemical mixture property prediction database to classify the embeddings outputs. The database may be generated by generating property predictions for theoretical chemical mixtures using an embedding model and a prediction model to determine predicted properties.

For example, the systems and methods can include obtaining molecule data for one or more molecules and mixture data associated with a mixture of the one or more molecules. The molecule data can include respective molecule data for each molecule of a plurality of molecules that make up a mixture. In some implementations, the mixture data can include data related to the concentration of each molecule in the mixture along with the overall composition of the mixture. The mixture data can describe the chemical formulation of the mixture. The molecule data can be processed with an embedding model to generate a plurality of embeddings. Each respective molecule data for each respective molecule may be processed with the embedding model to generate a respective embedding for each respective molecule in the mixture. In some implementations, the embeddings can include data descriptive of individual molecule properties for the embedded data. In some implementations, the embeddings can be vectors of numbers. In some cases, the embeddings may represent graphs or molecular property descriptions. The embeddings and the mixture data can be processed by a prediction model to generate one or more property predictions. The one or more property predictions can be based at least in part on the one or more embeddings and the mixture data. The property predictions can include various predictions on the taste, smell, coloration, etc. of the mixture. In some implementations, the systems and methods can include storing the one or more property predictions. In some implementations, one or both of the models can include a machine-learned model.

The embeddings and their respective property predictions can then be paired as a labeled set to generate labeled embeddings in the embedding space. The machine-learned model can be trained to output the embedding outputs that can then be compared to the labels in the embedding space for classification tasks such as determining the properties of a sensed chemical compound or for determining the chemical mixture sensed by the sensor.

The systems and methods of the present disclosure provide a number of technical effects and benefits. As one example, the system and methods can provide devices and processes that can enable the understanding and interpretation of electrical signals, which can lead to efficient and accurate identification processes. The systems and methods can further be used to identify spoilage of food with electrical sensors or the identification of plant, animal, or human disease states. Furthermore, the systems and methods can enable automated processes for chemical compound identification based on electrical signal data generated by an electronic chemical sensor.

Another technical benefit of the systems and methods of the present disclosure is the ability to leverage an odor embedding space for classification of the electrical signals. Manually training a model to identify every known mixture or property can be tedious, but the use of a generated odor embedding space can provide readily accessible data without having to start training from scratch.

Another example technical effect and benefit relates to improved computational efficiency and improvements in the functioning of a computing system. For example, certain existing systems are trained to identify the presence of a single chemical compound or a handful of compounds. Individually training for each compound can be time consuming, but it can also lead to computational inefficiencies when the system is only testing if the compound exists or doesn't exist. In contrast, by training a machine-learned model to generate an embedding output in an embedding space, the system can leverage embedding properties to efficiently determine chemical compounds or chemical properties. Therefore, the proposed systems and methods can save computational resources such as processor usage, memory usage, and/or network bandwidth.

With reference now to the Figures, example embodiments of the present disclosure will be discussed in further detail.

### Example Devices and Systems

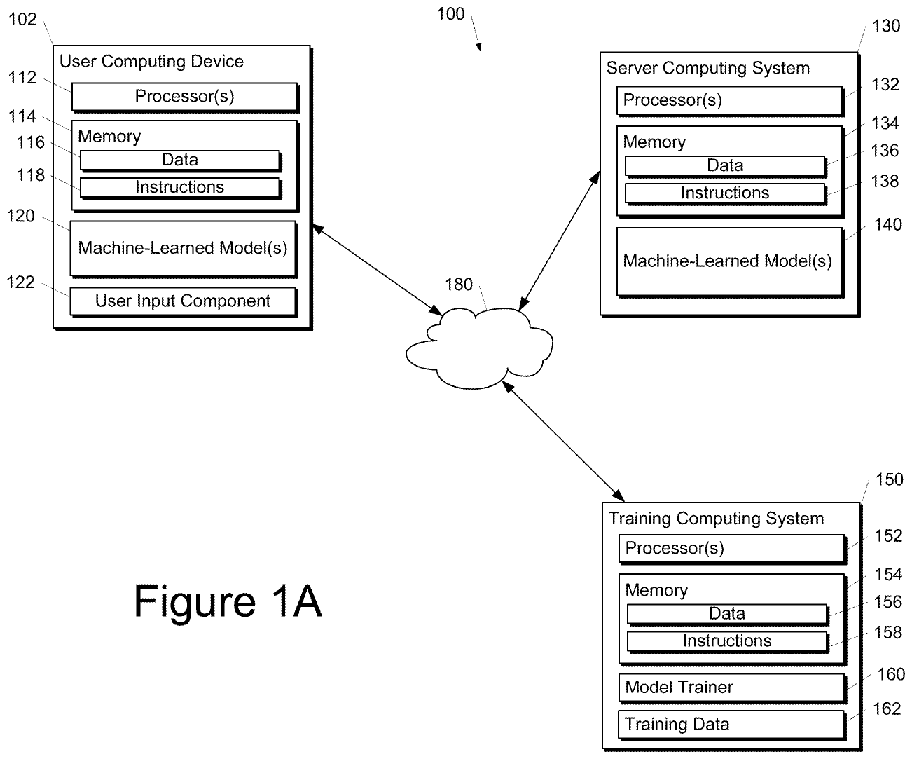

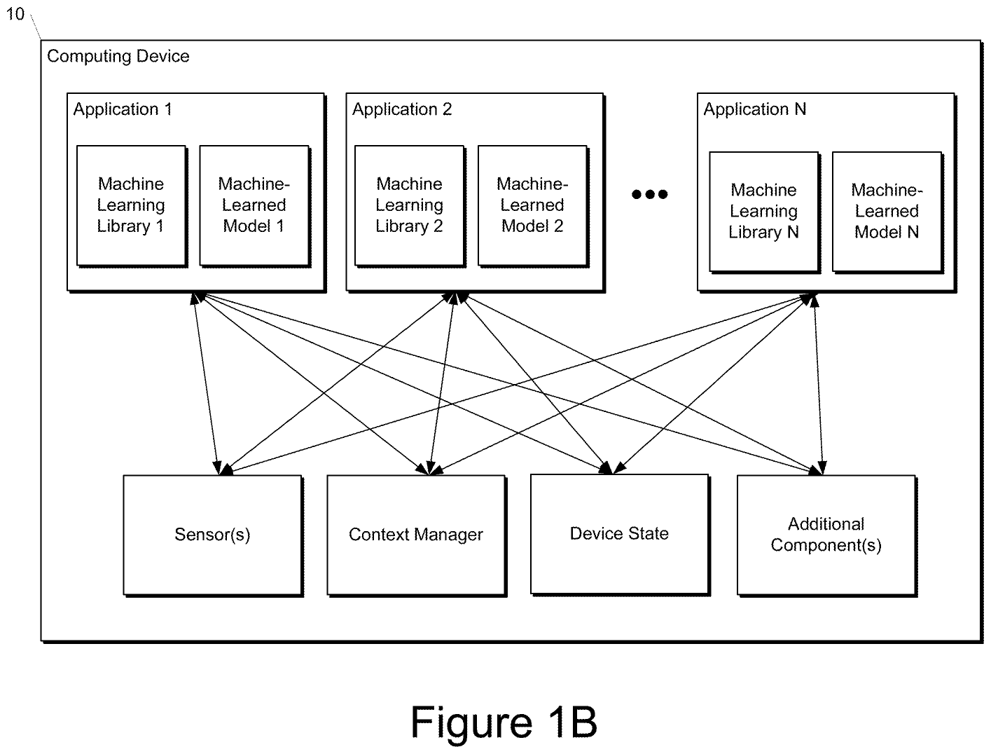

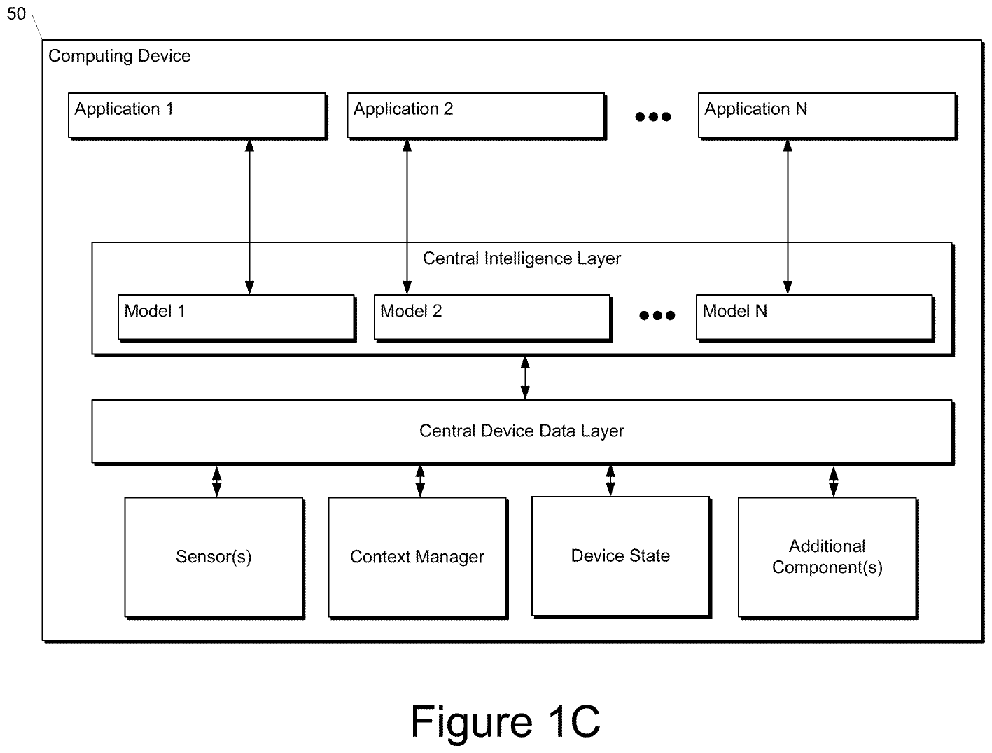

FIG. 1A depicts a block diagram of an example computing system 100 that performs electrical signal processing according to example embodiments of the present disclosure. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

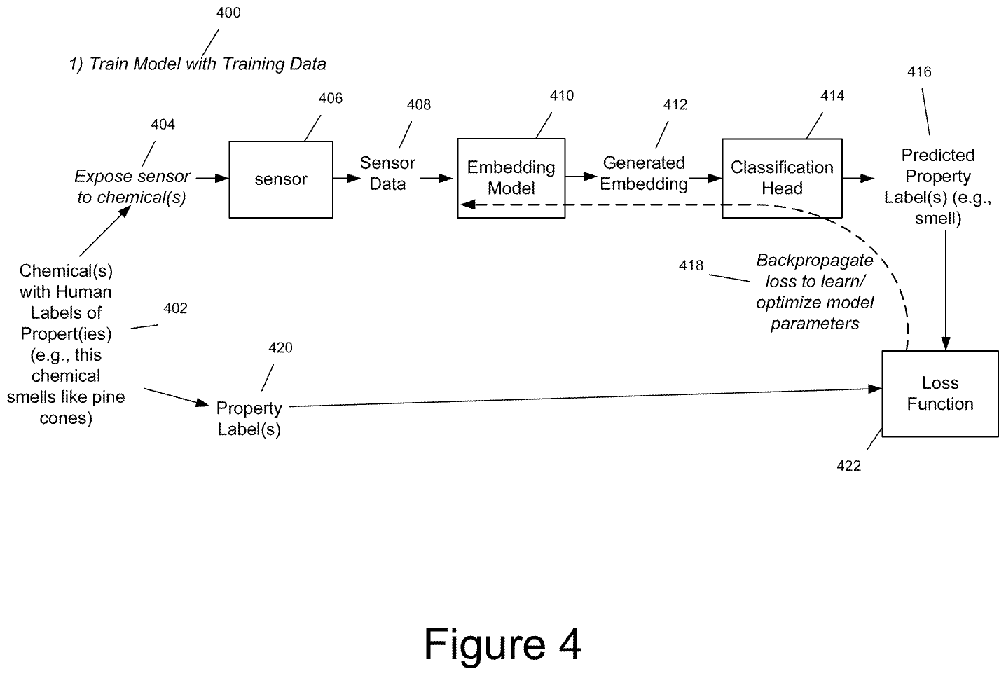

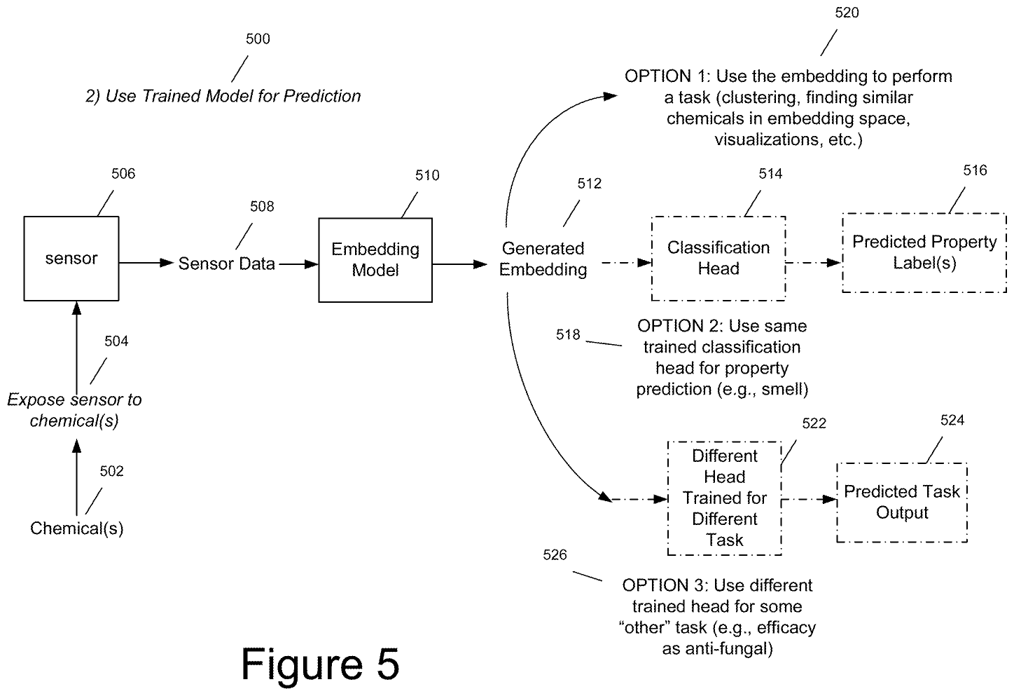

In some implementations, the user computing device 102 can store or include one or more electrical signal processing models 120. For example, the electrical signal processing models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Example electrical signal processing models 120 are discussed with reference to FIGS. 4, 5, & 9.

In some implementations, the one or more electrical signal processing models 120 can be received from the server computing system 130 over network 180, stored in the user computing device memory 114, and then used or otherwise implemented by the one or more processors 112. In some implementations, the user computing device 102 can implement multiple parallel instances of a single electrical signal processing model 120 (e.g., to perform parallel electrical signal processing across multiple instances of different chemical compounds being sensed).

More particularly, the electrical signal processing model can be a machine-learned model trained to receive sensor data descriptive of electrical signals indicative of a chemical compound, process the sensor data, and output an embedding output in an embedding space. The embedding output can then be used to perform a variety of tasks. For example, the embedding output may be processed with a classification model to determine the chemical compound molecules and concentration or the properties of the chemical compound. The results can then be provided to a user.

Additionally or alternatively, one or more electrical signal processing models 140 can be included in or otherwise stored and implemented by the server computing system 130 that communicates with the user computing device 102 according to a client-server relationship. For example, the electrical signal processing models 140 can be implemented by the server computing system 140 as a portion of a web service (e.g., an electronic chemical sensor service). Thus, one or more models 120 can be stored and implemented at the user computing device 102 and/or one or more models 140 can be stored and implemented at the server computing system 130.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned electrical signal processing models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Example models 140 are discussed with reference to FIGS. 4, 5, & 9.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the electrical signal processing models 120 and/or 140 based on a set of training data 162. The training data 162 can include, for example, paired sets of data in which each paired set includes electrical signal training data and a ground truth training label for the respective electrical signal training data.

In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

FIG. 1A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

FIG. 1B depicts a block diagram of an example computing device 10 that performs according to example embodiments of the present disclosure. The computing device 10 can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device 50 that performs according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

### Example Model Arrangements

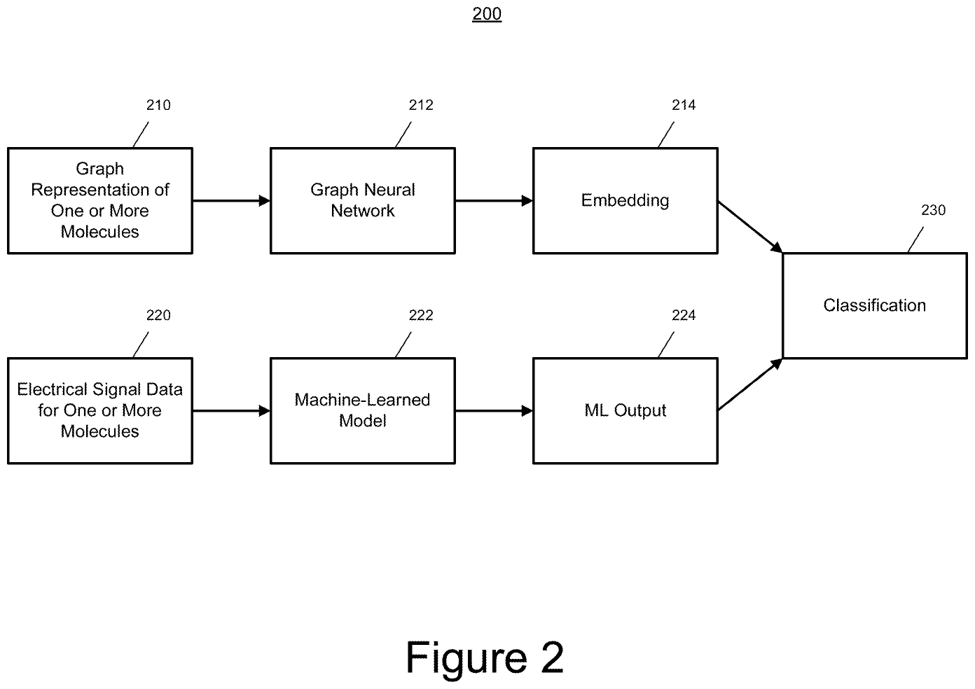

FIG. 2 depicts a block diagram of an example two footed classification system 200 according to example embodiments of the present disclosure. In some implementations, the two footed classification system 200 is trained to receive either graph-representations 210 of chemical compounds or electrical signal data 220 descriptive of a chemical compound and, as a result of receipt of the input data 210 & 220, provide output data 230 that classifies the input data as relating to the particular chemical compound or particular properties. Thus, in some implementations, the two footed classification system 200 can include a graph neural network 212 that is operable to process the graph representations 210, and a machine-learned model 222 that is operable to process the electrical signal data 220.

In particular, FIG. 2 depicts a system 200 that can provide a classification by processing either sensor data or graph representation data. The depicted system 200 includes a first foot for processing graph representations for one or more molecules 210, and a second foot for processing electrical signal data, or sensor data, for one or more molecules 220. However, in some implementations, a single model architecture can process both graph representations 210 and sensor data 220.

Processing of the graph representations 210 can include processing data descriptive of the graph representations 210 with a graph neural network (GNN) model 212 to generate an embedding 214. The embedding may be based at least in part on molecule concentrations. The embedding 214 can be an embedding in an embedding space.

Processing of the electrical signal data 220 can include processing the electrical signal data 220 with a machine-learned model 222 to generate a ML output 224. In some implementations, the electrical signal data 220 may be obtained from or generated with one or more sensors. The one or more sensors can include an electronic chemical sensor. Moreover, in some implementations, the electrical signal data 220 can include sensor data descriptive of one or more electrical signals generated in response to exposure to a chemical compound. The machine-learned model 222 can include one or more embedding models and/or one or more transformer models. Moreover, the ML output 224 can be an embedding output in an embedding space.

In some implementations, the GNN model 212 and the machine-learned model 22 can be trained to provide embeddings 214 and embedding outputs 224 in the same embedding space. Moreover, in some implementations, the GNN model 212 and the machine-learned model 222 may be a singular shared model. The two models may be part of the same model architecture.

The embeddings 214 and ML outputs 224 can then be processed with a classification model to determine a classification 230. The classification 230 can be based at least in part on a set of human-inputted labels. In some implementations, the classification 230 can be based at least in part on property prediction labels in the embedding space. The property prediction labels may be based at least in part on a chemical mixture property prediction system that utilizes an embedding model and a prediction model to determine property predictions of theoretical mixtures.

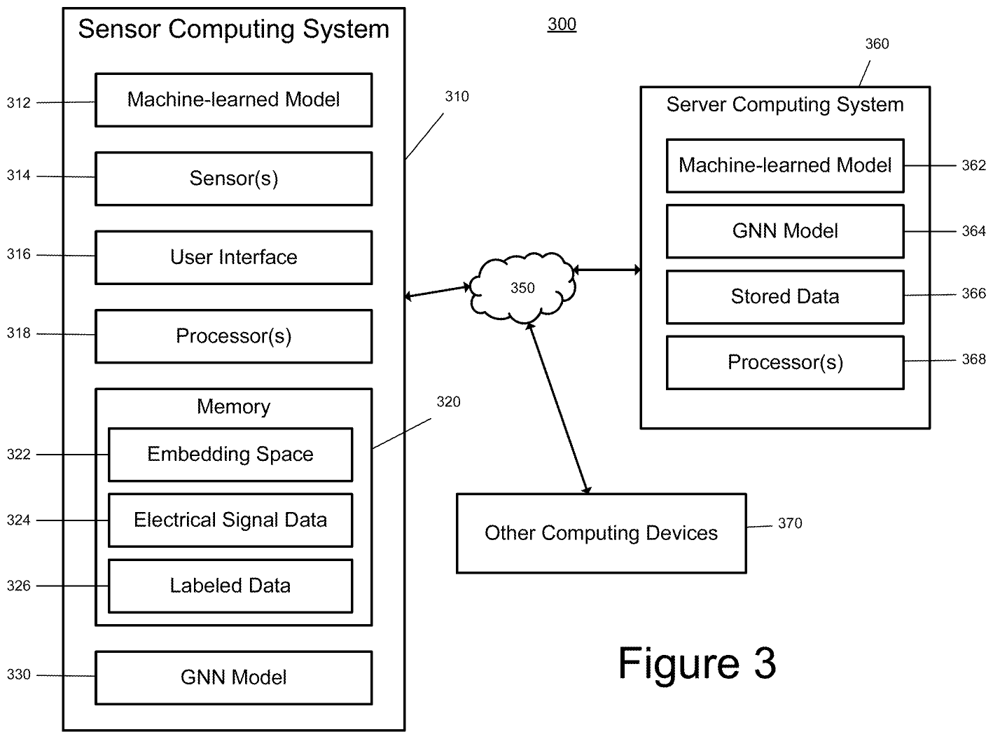

FIG. 3 depicts a block diagram of an example electronic chemical sensor device system 300 according to example embodiments of the present disclosure. In some implementations, the electronic chemical sensor device system 300 can include a sensor computing system 310 with a machine-learned model 312, one or more sensors 314, a user interface 316, processors 318, memory 320, and a GNN embedding model 330.

In particular, the sensor computing system 310 can include an electronic chemical sensor device including one or more sensors 314 for sensing chemical compound exposure. The sensors 314 can be configured to generate sensor data descriptive of electrical signals obtained in response to exposure to one or more molecules.

Moreover, the sensor computing system 310 can include a machine-learned model 312 for processing the sensor data to generate an embedding output in the embedding space. The sensor computing system may further include an embedding model 330 for processing graph representations and/or for jointly training the machine-learned model 312 with a graph neural network embedding model 330.

In some implementations, the sensor computing system can include one or more memory components 320 for storing embedding space data 322, electrical signal data 324, labeled data sets 326, other data, and instructions for performing one or more operations or functions. In particular, the memory 320 may store embedding space data 322 generated using a database of embedding-label pairs. For example, the embedding space data 322 can include a plurality of paired sets including embeddings generated based on graph representations or sensor data and a respective paired label descriptive of a chemical mixture or property predictions. The embedding space data 322 may aid in classification tasks such as determining the chemical compound a sensor was exposed to.

The memory components may also store past electrical signal data 324 and labeled data 326. Past electrical signal data 324 can be stored for training, classification tasks, and/or for keeping a data log of past intake data. For example, a set of electrical signal data 324 may not reach a threshold classification score for any stored labels or classes and may therefore be stored as a new classification label or class. However, in some implementations, the electrical signal data 324 may match a classification threshold but contain a deviation value from the training data. The sensor computing system may log past electrical signal data 324 or past sensor data to determine reoccurring deviation trends or errors that may indicate a need for sensor calibration or parameter adjustment.

Alternatively and/or additionally, the memory components 320 may store labeled data sets 326 in place of or in combination with the embedding space data 322. The labeled data sets 326 can be utilized for classification tasks or for training the machine-learned model 312. In some implementations, the sensor computing system 310 may actively intake human-inputted labels for improving the accuracy of classification tasks or for future training.

The sensor computing system can include a user interface 316 intaking user inputs and for providing notifications and feedback to the user. For example, in some implementations, the sensor computing system 310 may include a display on or attached to the electronic chemical sensor that can display a user interface that provides notifications on embedding values, sensor data classifications, etc. In some implementations, the electronic chemical sensor can include a touch screen display for receiving inputs from a user to aid in use of the electronic chemical sensor.

The sensor computing system 310 can communicate with one or more other computing systems over a network 350. For example, the sensor computing system 310 can communicate with a server computing system 360 over the network 350. The server computing system 360) can include a machine-learned model 362, a graph neural network embedding model 364, stored data 366, and one or more processors 368. In some implementations, the server computing system 360 can receive sensor data or labeled data 326 from the sensor computing system in order to help retrain the machine-learned model or for diagnostic tasks. In some implementations, the server computing system's 360 stored data 366 can include a labeled embedding database that can be accessed by the sensor computing system 310 over the network to aid in classification tasks and training. In some implementations, the server computing system 360 can provide updated models to one or more sensor computing systems 310. Moreover, in some implementations, the sensor computing system 310 may utilize the one or more processors 368 and the machine-learned-model 362 of the server computing system 360 for processing sensor data generated by the one or more sensors 314.

In some implementations, the sensor computing system 370 can communicate with one or more other computing devices 370 for providing notifications, for processing sensor data from other computing devices 370, or for other computing tasks.

FIG. 4 depicts a block diagram of an example system for training a machine-learned model 400 according to example embodiments of the present disclosure. In some implementations, the system for training a machine-learned model 400 can involve training the machine-learned model 410 to receive a set of input data 404 descriptive of a chemical compound and, as a result of receipt of the input data 404, provide output data 416 that is descriptive of a predicted property label or chemical mixture label. Thus, in some implementations, the system for training a machine-learned model 400 can include a classification model 414 that is operable to classify the generated embeddings 412.

The machine-learned model can be trained using ground truth labels. In some implementations, the machine-learned model can be an embedding model 410 trained to process sensor data 408 to output a generated embedding output 412, which can then be used for a variety of other tasks.

In some implementations, training the embedding model 400 can begin with one or more training chemicals with human labels of properties 402. The one or more chemicals 404 can be exposed to one or more sensors 406 to generate sensor data descriptive of the exposure to the one or more chemicals 404. In some implementations, the sensor data can be descriptive of electrical signals (e.g., voltage or current) generated by an electronic chemical sensor.

The generated sensor data 408 can then be processed by an embedding model 410 to generate an embedding output 412. The embedding model 410 can include one or more transformer models. In some implementations, the embedding model 410 can include a graph neural network model and may be trained to be able to process both graph representations and sensor data 408. Moreover, the generated embedding 412 can be an embedding output in an embedding space, which can include a set of identifier values similar to RGB values for color display.

The generated embedding 412 can then be processed by a classification head 414 to determine one or more matching predicted property labels 416. The predicted property labels 416 can include sensory property labels such as smell, taste, or color. The predicted property labels 416 and the human inputted property labels 420 can then be used to evaluate a loss function 422. The loss function 422 can then be used to adjust one or more parameters of the machine-learned model 410 by backpropagating the loss to learn/optimize model parameters 418.

The process 400 can be completed iteratively for a plurality of training examples to train the machine-learned model 410 to generate embedding outputs 412 that can be used to perform classification tasks or perform other tasks based on obtained sensor data 408.

FIG. 5 depicts a block diagram of an example trained machine-learned model system 500 according to example embodiments of the present disclosure. In some implementations, the trained machine-learned model system 500 is trained to receive a set of input data 504 descriptive of a one or more chemicals and, as a result of receipt of the input data 504, provide output data 512 that includes a generated embedding. Thus, in some implementations, the trained machine-learned model system 500 can include a classification head 514 that is operable to determine predicted property labels 516.

The trained machine-learned model 510 can then be used for a variety of tasks including property prediction tasks.

For example, one or more chemicals 502 can be exposed 504 to one or more sensors 506 to generate sensor data 508. The one or more sensors 506 can include one or more electronic chemical sensors that can generate sensor data 508 descriptive electrical signal data observed during exposure to the one or more chemicals 502. Moreover, the one or more chemicals 502 may be exposed 504 to the one or more sensors 506 in a controlled environment (e.g., a lab space) or in an uncontrolled environment (e.g., a car, an office, etc.).

The sensor data 508 can then be processed by the trained embedding model 510 to generate an embedding output 512. The embedding output 512 can be an embedding in an embedding space and may include a plurality of values descriptive of vector values.

In some implementations, the embedding output 512 alone can be useful clustering similar chemicals based on embeddings generated from sensor data of different chemicals 520. The embedding outputs 512 can also be used for better understanding the embedding space and the properties of different chemicals in the embedding space. Alternatively and/or additionally, the embedding output alone can be utilized for a variety of tasks that can include generating a visualization of the embedding space to provide a more intuitive depiction of the chemical property space. The generated embedding output can be used for further model training or a variety of other tasks.

Other applications of the embedding output 512 can include classification tasks 518, which can include processing the embedding output 512 with a classification head 514 to determine one or more associated predicted property labels 516. The classification head 514 can be trained for property prediction tasks such as olfactory property prediction, which can be used to determine when a car needs to be serviced by a cleaning service or for determining when a bad odor is present.

Alternatively and/or additionally, the embedding output 512 can be processed by a different head trained for a different task 522 to provide a predicted task output 524 to aid in performing a task 524. In some implementations, the different head 522 can be trained to classify whether the embedding output is descriptive of food spoilage, a disease state, or whether the chemical might have beneficial properties such as an anti-fungal.

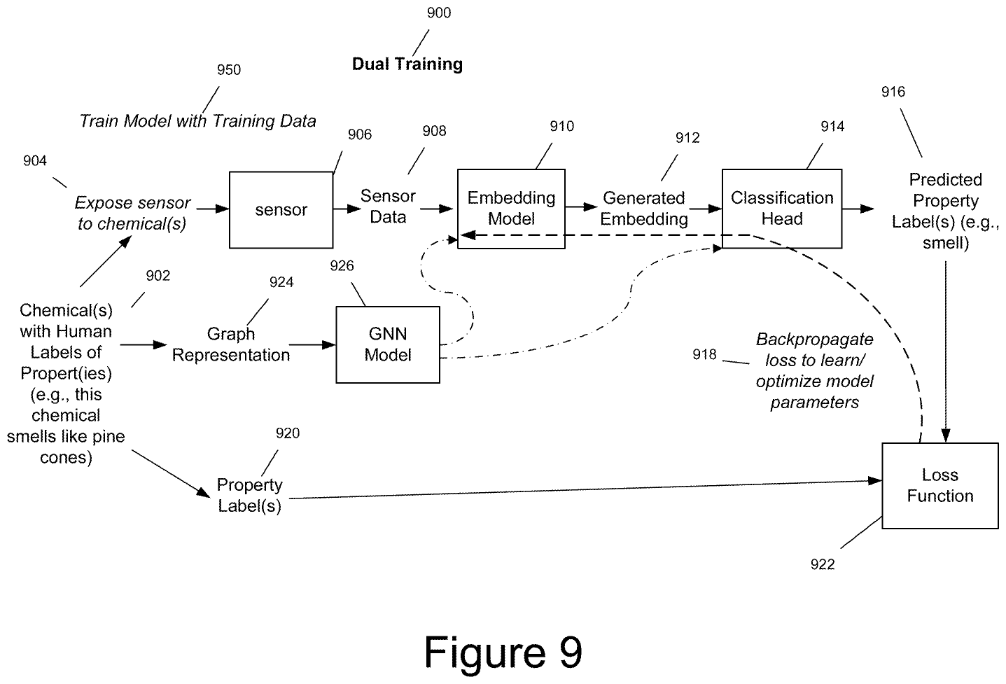

FIG. 9 depicts a block diagram of an example system for training a machine-learned model 900 according to example embodiments of the present disclosure. The system for training a machine-learned model 900 is similar to the system for training a machine-learned model 400 of FIG. 4 except that the system for training a machine-learned model 900 further includes training the system to process graph representations.

In some implementations, the machine-learned models 910 and 926 can be trained using ground truth labels. In some implementations, the machine-learned models can be embedding models 910 and 926 trained to process sensor data 908 and/or data descriptive of a graph representation 924 to output a generated embedding output 912, which can then be used for a variety of other tasks.

In some implementations, training the embedding models 900 can begin with one or more training chemicals with human labels of properties 902. The one or more chemicals 904 can be exposed to one or more sensors 906 to generate sensor data descriptive of the exposure to the one or more chemicals 904. In some implementations, the sensor data can be descriptive of electrical signals (e.g., voltage or current) generated by an electronic chemical sensor.

The generated sensor data 908 can then be processed by an embedding model 910 to generate an embedding output 912. The embedding model 910 can include one or more transformer models. In some implementations, the embedding model 910 can include a graph neural network model 926 and may be trained to be able to process both graph representations 924 and sensor data 908. Moreover, the generated embedding 912 can be an embedding output in an embedding space, which can include a set of identifier values similar to RGB values for color display.

In some implementations, the system can be a two-footed system that can process either sensor data 908 or data descriptive of a graph representation 924 to generate the embedding output 912. Moreover, in some implementations, a graph neural network model 926 and the embedding model 910 may be jointly trained. In some implementations, the graph representation data 924 may be processed by a graph neural network model 926 before being processed by the embedding model 910; however, in some implementations, the GNN model 926 may output an embedding that can be processed by the classification head 914 to determine predicted property labels 916 without be processed by the embedding model 910.

The generated embedding 912 can then be processed by a classification head 914 to determine one or more matching predicted property labels 916. The predicted property labels 916 can include sensory property labels such as smell, taste, or color. The predicted property labels 916 and the human inputted property labels 920 can then be used to evaluate a loss function 922. The loss function 922 can then be used to adjust one or more parameters of at least one of the machine-learned models 910 and/or 926 by backpropagating the loss to learn/optimize model parameters 918.

The process 900 can be completed iteratively for a plurality of training examples to train the machine-learned models 910 and 926 to generate embedding outputs 912 that can be used to perform classification tasks or perform other tasks based on obtained sensor data 908.

### Example Methods

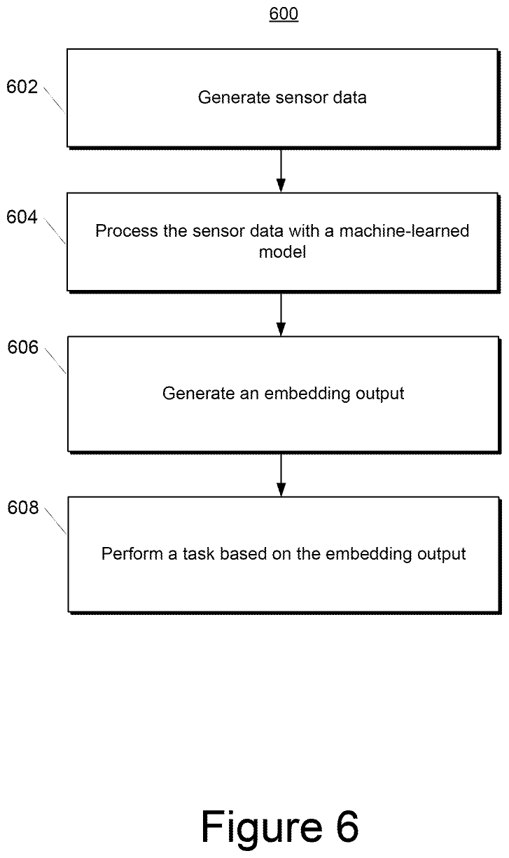

FIG. 6 depicts a flow chart diagram of an example method to perform according to example embodiments of the present disclosure. Although FIG. 6 depicts steps performed in a particular order for purposes of illustration and discussion, the methods of the present disclosure are not limited to the particularly illustrated order or arrangement. The various steps of the method 600 can be omitted, rearranged, combined, and/or adapted in various ways without deviating from the scope of the present disclosure.

At 602, a computing system can generate sensor data. The sensor data can be generated with one or more sensors, which can include an electronic chemical sensor. In some implementations, the sensor data may be descriptive of electrical signals (e.g., voltage or current) generated by the sensors in response to exposure to one or more molecules.

At 604, the computing system can process the sensor data with a machine-learned model. The machine-learned model can include one or more transformer models and/or one or more GNN embedding models. Moreover, the machine-learned model can be a machine-learned model trained to process sensor data to generate embedding outputs in an embedding space.

At 606, the computing system can generate an embedding output. The embedding output can include one or more values similar to RGB values for color display.

At 608, the computing system can perform a task based on the embedding output. For example, the embedding output can be processed by a classification model to determine the sensed chemical or the properties of the sensed chemical. Classifying the embedding output can involve the use of labeled embeddings in the embedding space, training examples, or other classification techniques. In some implementations, the embedding output can be processed by a classification head to determine sensory properties of the sensed chemical (e.g., smell, taste, color, etc.). In other implementations, the classification head may be trained to identify a disease state based on the embedding output. The embedding output may be used to enable sensor devices to identify food spoilage, diseased crops, bad odors, etc. in real-time.

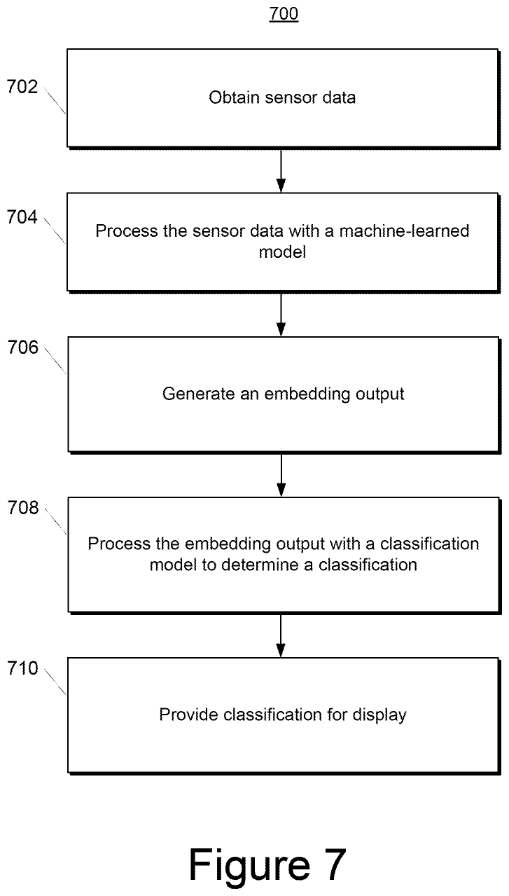

FIG. 7 depicts a flow chart diagram of an example method to perform according to example embodiments of the present disclosure. Although FIG. 7 depicts steps performed in a particular order for purposes of illustration and discussion, the methods of the present disclosure are not limited to the particularly illustrated order or arrangement. The various steps of the method 700 can be omitted, rearranged, combined, and/or adapted in various ways without deviating from the scope of the present disclosure.

At 702, a computing system can obtain sensor data. Sensor data can be obtained with one or more sensors and can be descriptive of an exposure to one or more molecules.

At 704, the computing system can process the sensor data with a machine-learned model. The machine-learned model can include one or more embedding models trained to process sensor data descriptive of raw electrical signal data to generate embedding outputs.

At 706, the computing system can generate an embedding output.

At 708, the computing system can process the embedding output with a classification model to determine a classification. The classification model can include one or more classification heads trained to identify one or more matching labels in an embedding space. In some implementations, the classification model may determine an associated label for the embedding output based on a threshold similarity determined at least in part on the embedding output's values or the embedding output's location in the embedding space.

At 710, the computing system can provide a classification for display. The classification may be a chemical mixture identification, one or more property predictions, or another form of classification (e.g., a disease state classification, food spoilage classification, a ripeness classification, bad odor classification, diseased crop classification, etc.). The display may include an LED display, an LCD display, an ELD display, a plasma display, a QLED display, or one or more lights affixed above labels. In some implementations, the classification may be displayed along with a visual representation of the embedding output in the embedding space. Moreover, in some implementations, similarity scores for different classifications may be displayed. If a threshold is not met for any classification, the system may display the closest classes along with similarity scores.

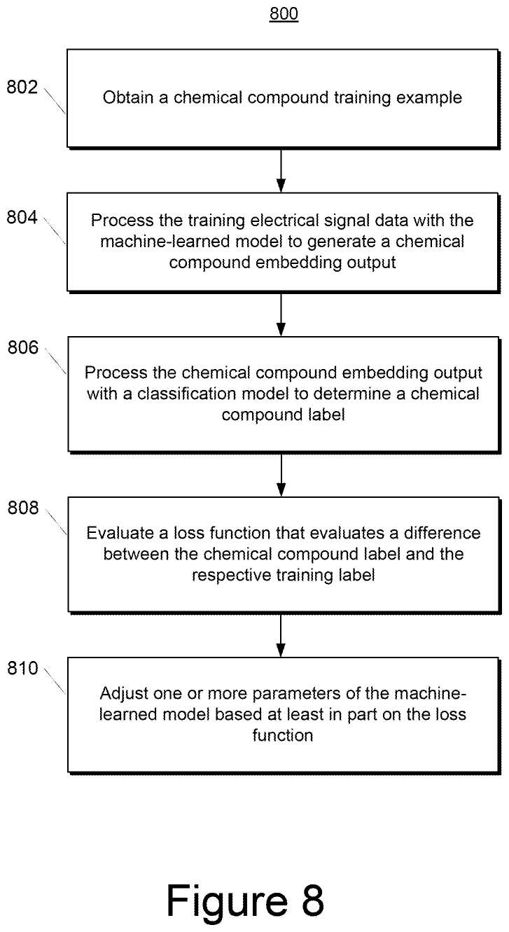

FIG. 8 depicts a flow chart diagram of an example method to perform according to example embodiments of the present disclosure. Although FIG. 8 depicts steps performed in a particular order for purposes of illustration and discussion, the methods of the present disclosure are not limited to the particularly illustrated order or arrangement. The various steps of the method 800 can be omitted, rearranged, combined, and/or adapted in various ways without deviating from the scope of the present disclosure.

At 802, a computing system can obtain a chemical compound training example. The chemical compound training example can include electrical signal training data and a respective training label. The electrical signal training data and the respective training label can be descriptive of a specific training chemical compound.

At 804, the computing system can process the training electrical signal data with the machine-learned model to generate a chemical compound embedding output. The chemical compound embedding output can include an embedding in an embedding space.

At 806, the computing system can process the chemical compound embedding output with a classification model to determine a chemical compound label. The classification model can be trained to identify one or more associated chemical compound labels. In some implementations, the classification model can include one or more classification heads trained for specific classifications.

At 808, the computing system can evaluate a loss function that evaluates a difference between the chemical compound label and the respective training label.

At 810, the computing system can adjust one or more parameters of the machine-learned model based at least in part on the loss function.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

What is claimed is:

1. A computing system comprising:
a sensor configured to generate electrical signals indicative of presence of one or more chemical compounds in an environment;
a machine-learned model trained to receive and process the electrical signals to generate an embedding in an embedding space;
wherein the machine-learned model has been trained using a training dataset comprising a plurality of training examples, each training example comprising a ground truth property label applied to a set of electrical signals generated by one or more test sensors when exposed to one or more training chemical compounds, each ground truth property label descriptive of a property of the one or more training chemical compounds;
one or more processors; and
one or more non-transitory computer-readable media that collectively store instructions that, when executed by the one or more processors, cause the computing system to perform operations, the operations comprising:
generating, by the sensor, sensor data indicative of presence of a specific chemical compound in the environment; and
processing, by the one or more processors, the sensor data with the machine-learned model to generate an embedding output in the embedding space.

2. The computing system of any preceding claim, further comprising: performing a task based on the embedding output.

3. The computing system of any preceding claim, wherein the task comprises providing a sensory property prediction based on the embedding output.

4. The computer system of any preceding claim, wherein the task comprises providing an olfactory property prediction based on the embedding output.

5. The computing system of any preceding claim, wherein the task is identifying a disease state based at least in part on the embedding output.

6. The computing system of any preceding claim, wherein the task is determining a malodor state based at least in part on the embedding output.

7. The computing system of any preceding claim, wherein the task is determining if spoilage has occurred based at least in part on the embedding output.

8. The computing system of any preceding claim, wherein the task comprises providing a human-inputted label for display, wherein the human-inputted label is determined by an association with the embedding output in the embedding space.

9. The computing system of claim 8, wherein the human-inputted label is descriptive of a name of a particular food.

10. The computing system of any preceding claim, wherein the machine-learned model is trained jointly with a graph neural network, wherein training comprises: jointly training the machine-learned model and the graph neural network to generate a single, combined output within the embedding space.

11. The computing system of claim 10, wherein the graph neural network is trained to receive a graph-based representation of the specific chemical compound as an input and output a respective embedding in the embedding space.

12. The computing system of any preceding claim, wherein the machine-learned model has been trained by:
obtaining a chemical compound training example comprising electrical signal training data and a respective training label, wherein the electrical signal training data and the respective training label are descriptive of a specific training chemical compound;
processing the electrical signal training data with the machine-learned model to generate a chemical compound embedding output;
processing the chemical compound embedding output with a classification model to determine a chemical compound label;
evaluating a loss function that evaluates a difference between the chemical compound label and the respective training label; and
adjusting one or more parameters of the machine-learned model based at least in part on the loss function.

13. The computing system of any preceding claim, wherein the machine-learned model is trained with supervised learning.

14. The computing system of any preceding claim, wherein the sensor data is descriptive of at least one of voltage or current.

15. The computing system of any preceding claim, wherein the machine-learned model comprises a transformer model.

16. The computing system of any preceding claim, further comprising: storing the embedding output.

17. The computing system of any preceding claim, wherein the sensor data is descriptive of an amplitude of one or both of voltage or current for one or more electrical signals.

18. The computing system of any preceding claim, wherein processing, by the one or more processors, the sensor data with the machine-learned model to generate the embedding output in the embedding space comprises: compressing the sensor data to a fixed length vector representation.

19. A computer-implemented method, the method comprising:
obtaining, by a computing system comprising one or more processors, sensor data with one or more sensors, wherein the sensor data is descriptive of electrical signals generated due to a presence of one or more chemical compounds in an environment;
processing, by the computing system, the sensor data with a machine-learned model to generate an embedding output in an embedding space, wherein the machine-learned model is trained to receive and process data descriptive of electrical signals to generate an embedding in the embedding space;
determining, by the computing system, one or more labels associated with the embedding output in the embedding space; and
providing, by the computing system, the one or more labels for display.

20. One or more non-transitory computer readable media that collectively store instructions that, when executed by one or more processors, cause a computing system to perform operations, the operations comprising:
obtaining sensor data with one or more sensors, wherein the sensor data is descriptive of electrical signals generated due to the presence of one or more chemical compounds in an environment;
processing the sensor data with a machine-learned model to generate an embedding output in an embedding space, wherein the machine-learned model is trained to receive and process data descriptive of electrical signals to generate an embedding in the embedding space;
obtaining a plurality of stored sensory property data sets, wherein the plurality of stored sensory property data sets comprises stored embeddings in the embedding space paired with a respective sensory property data set associated with the respective stored embedding;
determining one or more sensory properties based on the embedding output in the embedding space and the plurality of stored sensory property data sets; and
providing the one or more sensory properties for display.
